(19) United States
(12) Patent Application Publication
(10) Pub. No.: US 2021/0282389 A1
(43) Pub. Date: Sep. 16, 2021

(54) METHODS AND SYSTEMS FOR SAMPLE COLLECTION

(71) Applicant: OneGevity Health, LLC, New York, NY (US)

(72) Inventors: Hui Hua, New York, NY (US); Joel Dudley, New York, NY (US); Bodi Zhang, New York, NY (US); Christopher E. Mason, New York, NY (US)

(21) Appl. No.: 17/107,701

(22) Filed: Nov. 30, 2020

Related U.S. Application Data
(63) Continuation of application No. PCT/US19/35036, filed on May 31, 2019.
(60) Provisional application No. 62/679,695, filed on Jun. 1, 2018.

(51) Int. Cl. A01N 1/02

(52) U.S. Cl. CPC A01N 1/0231; C08L 29/04

(57) ABSTRACT

Presented herein are methods and systems of collecting and preserving a biological sample. The methods and systems include the use of a dissolvable collection film to collect the samples and dissolving the film in to a buffer or stabilizing solution contained in a sealable container.

### CROSS REFERENCE TO RELATED APPLICATIONS

This application is a continuation application of International Application No. PCT/US19/035036, filed May 31, 2019, which claims the benefit of U.S. provisional patent application 62/679,695, filed Jun. 1, 2018, the contents of which are hereby incorporated by reference in their entirety.

### BACKGROUND

Detection and identification of biomolecules such as microbiomes and metabolites from samples is widely used for diagnosis and monitoring of diseases. Stable transportation and delivery of biomolecules is generally required for such analysis. Low cost and efficient collection, storage and delivery of biomolecules are pertinent for the field of medical diagnosis. Presented herein are methods and systems to collect and stabilize a biological sample and methods to efficiently process the collected sample.

### SUMMARY

In some aspects, provided herein are methods of preserving a biological sample, comprising collecting a biological sample on a dissolvable film (e.g., a water-soluble film, an ethanol-soluble film, etc.); and dissolving the dissolvable film including the biological sample in a stabilizing solution, wherein the stabilizing solution is disposed in a sealable container. In some embodiments, the method further comprises analyzing the biological sample.

In some embodiments, the biological sample comprises a microbiome population. In some embodiments, the biological sample comprises a metabolite population. In some embodiments, the biological sample comprises a fecal, skin, vaginal, ear, or oral sample.

In some embodiments, the dissolvable film comprises a polyvinyl alcohol polymer. In some embodiments, the dissolvable film comprises cellulose. In some embodiments, the dissolvable film comprises a polyvinyl butyral (PVB) polymer. In some embodiments, the dissolvable film comprises from 1% to 30% PVB by weight. In some embodiments, the dissolvable film comprises from 30% to 50% PVB by weight. In some embodiments, the dissolvable film comprises from 50% to 100% PVB by weight. In some embodiments, the dissolvable film comprises a PVB and a polymer plasticizer (e.g., a combination of a PVB and a plasticizer). In some embodiments, the dissolvable film comprises a combination of a polyvinyl alcohol polymer, a polymer compatibilizer, and a polymer plasticizer or a sugar alcohol plasticizer. In some embodiments, the polymer compatibilizer comprises cellulose ether polymer, sodium carboxymethyl cellulose, modified starch, or a combination thereof. In some embodiments, the dissolvable film comprises from 10% to 30% by weight of a polymer compatibilizer and a polymer plasticizer or a sugar alcohol plasticizer. In some embodiments, the dissolvable film comprises from 1% to 30% polyvinyl alcohol polymer by weight. In some embodiments, the dissolvable film comprises from 30% to 50% polyvinyl alcohol polymer by weight. In some embodiments, the dissolvable film comprises from 50% to 100% polyvinyl alcohol polymer by weight. In some embodiments, the dissolvable film is substantially soluble in the stabilizing solution below 37° C. In some embodiments, the dissolvable film is edible, food grade, cosmetic grade, pharmaceutical grade, or suitable for skin contact.

In some embodiments, the stabilizing solution comprises a preservative or a stabilizing reagent. In some embodiments, the preservative or the stabilizing reagent is a high salt reagent or an ethanol-based reagent. In some embodiments, the stabilizing solution comprises an antiseptic reagent. In some embodiments, the stabilizing solution comprises N-octylpyridinium bromide solution. In some embodiments, the stabilizing solution comprises lithium chloride, tris aminomethane, ethanol, TCEP-HCl, or a combination thereof.

In some embodiments, a volume of the sealable container is greater than 5 ml. In some embodiments, a volume of the sealable container is less than 150 ml.

In some embodiments, the stabilizing solution stabilizes the biological sample at room temperature for up to 15 days. In some embodiments, the stabilizing solution stabilizes the nucleic acid content of the biological sample at room temperature for up to 30 days or at −20° C. for up to 3 months. In some embodiments, the stabilizing solution stabilizes the protein content of the biological sample at room temperature for up to 15 days. In some embodiments, the stabilizing solution stabilizes the metabolites of the biological sample at room temperature for up to 7 days or at −80° C. for up to 3 months.

In some embodiments, the method further comprises transporting or delivering the sealable container comprising the stabilizing solution and the biological sample to a laboratory or a testing center. In some embodiments, the sealable container is transported or delivered at room temperature. In some embodiments, the sealable container is transported or delivered on dry ice, in an ice box, in a cool box (e.g., a NanoCool® box), or with an ice pack. In some embodiments, the sealable container comprising the stabilizing solution and the biological sample is not frozen before any further processing.

In some embodiments, the surface area of the dissolvable film is from 3 cm×3 cm to 40 cm×40 cm.

In some aspects, provided herein are sampling kits comprising: a dissolvable film for the collection of a biological sample and a stabilizing solution in a sealable container, the stabilizing solution capable of dissolving the dissolvable film including the biological sample.

In some embodiments, the kit further comprises instructions for collecting the biological sample onto the dissolvable film and dissolving the dissolvable film in the stabilizing solution.

In some embodiments, the dissolvable film is individually packaged. In some embodiments, the dissolvable film is provided as a stacked set of sheets in a package. In some embodiments, the dissolvable film comprises polyvinyl alcohol. In some embodiments, the dissolvable film comprises polyvinyl butyral. In some embodiments, the dissolvable film is dry.

In some embodiments, the sealable container is barcoded. In some embodiments, the stabilizing solution comprises DMSO-disodium EDTA-saturated salt (DESS). In some embodiments, the stabilizing solution comprises ethanol.

In some aspects, provided herein are methods for identifying a microbiome population from a solution, the methods comprising: performing a nucleic acid content analysis on a stabilizing solution comprising a biological sample and a dissolved dissolvable film and identifying at least one microbe present in the stabilizing solution comprising the biological sample based on the nucleic acid content analysis. In some embodiments, the biological sample is collected onto the dissolvable film and the dissolvable film is dissolved in the stabilizing solution prior to the performing of the nucleic acid content analysis.

In some embodiments, the dissolvable film can be a water-soluble film, and the stabilizing solution can be water based, such that the water-soluble film is dissolvable in the water based stabilizing solution. In some embodiments, the dissolvable film can be an ethanol-soluble film, and the stabilizing solution can be ethanol based, such that the ethanol-soluble film is dissolvable in the ethanol based stabilizing solution.

Some embodiments further comprise determining a microbiome profile of the biological sample. In some embodiments, determining the microbiome profile comprises identifying at least five microbes, at least 10 microbes, at least 20 microbes, or at least 30 microbes in the biological sample.

Some embodiments further comprise generating a report identifying at least one microbe present in the microbiome population.

In some embodiments, the nucleic acid content analysis comprises sequencing the nucleic acid content of the biological sample. In some embodiments, sequencing the nucleic acid content comprises next-generation sequencing, long-read sequencing, Sanger sequencing, high-throughput sequencing, fluorescence-based sequencing, polymerase chain reaction based sequencing, or nanopore sequencing. In some embodiments, sequencing the nucleic acid content comprises sequencing the 16S V1-V2, V3-V4, or V4-V5 sequences for bacteria; ITS1 region for fungi; or 18S region for eukaryotic microbes. In some embodiments, sequencing the nucleic acid content comprises sequencing the whole genome of a microbe.

In some embodiments, identifying the microbiome population does not comprise freezing the biological sample. In some embodiments, identifying the microbiome population does not comprise a thawing step. In some other embodiments, identifying the microbiome population comprises a thawing step.

In some embodiments, the stabilizing solution comprising the biological sample and the dissolved dissolvable film further comprises intact cells and non-intact cells. In some embodiments, the stabilizing solution comprising the biological sample and the dissolved dissolvable film further comprises non-intact cells.

In some embodiments, the method further comprises purification of nucleic acid content. In some embodiments, the purification of nucleic acid content is performed using a nucleic acid purification kit.

In some aspects, provided herein are methods for identifying a metabolite population from a solution, the methods comprising: performing a protein or lipid content analysis on a stabilizing solution including a biological sample and a dissolved dissolvable film and identifying at least one metabolite present in the solution comprising the biological sample based on the protein or lipid analysis.

In some embodiments, the biological sample is collected onto the dissolvable film and the dissolvable film is dissolved in the stabilizing solution prior to the performing of the protein or lipid content analysis.

In some aspects, provided herein are systems for analyzing a biological sample, the systems comprising: a sampling kit including a dissolvable film for the collection of the biological sample and a sealable container comprising a stabilizing solution for dissolving the dissolvable film and a sample processing module configured to identify at least one microbe or metabolite in the biological sample.

In some embodiments, the sample processing module comprises an immunoassay, a flow cytometer, a microchip, a sequencer, LC/MS, GC/MS, or NMR. In some embodiments, the sample processing module is configured to identify at least one microbe or metabolite by detecting or measuring a level of the microbe or the metabolite. In some embodiments, the sample processing module is configured to identify at least 5 microbes or metabolites, at least 10 microbes or metabolites, at least 20 microbes or metabolites, or at least 50 microbes or metabolites.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a flow chart depicting a process for determining quality of a sample collected by a method described herein, followed by cluster preparation, sequencing, and bioinformatics.

FIG. 2 shows at the phylum level the relative abundance of microbial species present in samples by a method described herein or by a conventional method.

FIG. 3 shows a taxonomic composition distribution at the class level of microbial species present in samples by a method described herein or by a conventional method.

FIG. 4 shows a taxonomic composition distribution at the order level of microbial species present in samples by a method described herein or by a conventional method.

### DETAILED DESCRIPTION

Identification of a microbiome or metabolite population from a subject, such as a human subject can aid in assessing the subject's health. There are numerous host and environmental factors that can influence the microbiome and metabolite population in a subject. Additionally, to identify biomolecules in a sample, the sample can be preserved in a stable manner so as to decrease or inhibit degradation of biomolecules such as nucleic acid content over time. Collection, preservation, transportation and delivery of such biomolecules can play a role in accurate analysis of the microbiome and metabolite population. Preservation of biomolecules can also assist in situations such as sampling in remote areas where cryopreservation may not be possible and may help reduce the costs for such analysis.

The present disclosure contemplates methods and systems for collecting and preserving biological samples such as cells, metabolites, nucleic acid molecules, proteins, carbohydrates and lipids in an efficient, cost-effective and/or ecological manner. Contemplated herein is the use of a dissolvable film for the collection of a biological sample for further processing. The methods and systems of the present disclosure also contemplate convenient storage and transportation of a biological sample without affecting or substantially affecting sample integrity.

Such biological samples are often derived from a subject, such as a human, and can be useful as biomarkers for identifying the microbiome and metabolite population in a biological sample. The biological sample can refer to a sample of a subject. The biological sample can comprise any number of cells, for example, microbes and a subject's cells. The biological sample can also comprise macromolecules such as cellular macromolecules. The biological sample can comprise nucleic acid material, metabolites, proteins, lipids or carbohydrates.

The biological sample can be derived from a fluid sample, such as urine sample, vaginal sample, oral sample or saliva sample. The biological sample can also be derived from a solid sample, such as a skin sample, stool sample or ear sample. The biological sample can be a cell or a cell-free sample. The biological sample can comprise nucleic acid content, non-limiting examples of which include DNA, RNA, genomic DNA, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), viral RNA, ribozymes, and cDNA.

Use of the film material disclosed herein can allow for the collection of the sample. Alternatively, use of the film material disclosed herein can provide for the maintenance of integrity of macromolecules in the sample. Such macromolecules can include, for example, nucleic acids, protein, lipids and metabolite content.

### Collection of a Biological Sample Using a Dissolvable Film

The biological sample, or elements thereof, can be collected using a dissolvable film. A sample can be collected from its natural environment using such a film. For instance, the dissolvable film can be used to collect a skin sample, a vaginal sample, a fecal sample or any other suitable sample. Alternatively, a collected sample can be transferred to a film of the disclosure. For instance, a saliva sample or a blood sample can be transferred to the film. The dissolvable film may be in the form of a wipe, napkin, tissue paper or swab.

The dissolvable film can be water soluble or ethanol soluble. In some cases, the dissolvable film can be soluble in a water based solution or in a mixture comprising ethanol. The dissolvable film can be soluble in other stabilizing or buffer solutions. Non-limiting examples of such solutions include stabilizing buffers, cell lysis buffers, ethanol-based buffers, etc. The stabilizing solution can be provided in a sealable container. In some cases, the dissolvable film can be dissolved in any suitable solvent, such as acetone, chloroform, isopropanol, methanol, diethyl ether, hexanes, ethanol, water, or a combination thereof. In some cases, the solvent can additionally comprise salt, acid, detergent, or another substance.

A dissolvable film can comprise a polyvinyl butyral (PVB). The dissolvable film can comprise 1-100% PVB. The dissolvable film can comprise at least 1% PVB. In some cases, the dissolvable film can comprise up to 100% PVB. In some cases, the dissolvable film can comprise 1% to 5% PVB, 1% to 10% PVB, 1% to 25% PVB, 1% to 50% PVB, 1% to 75% PVB, 1% to 80% PVB, 1% to 90% PVB, 1% to 95% PVB, 1% to 100% PVB, 5% to 10% PVB, 5% to 25% PVB, 5% to 50% PVB, 5% to 75% PVB, 5% to 80% PVB, 5% to 90% PVB, 5% to 95% PVB, 5% to 100% PVB, 10% to 25% PVB, 10% to 50% PVB, 10% to 75% PVB, 10% to 80% PVB, 10% to 90% PVB, 10% to 95% PVB, 10% to 100% PVB, 25% to 50% PVB, 25% to 75% PVB, 25% to 80% PVB, 25% to 90% PVB, 25% to 95% PVB, 25% to 100% PVB, 50% to 75% PVB, 50% to 80% PVB, 50% to 90% PVB, 50% to 95% PVB, 50% to 100% PVB, 75% to 80% PVB, 75% to 90% PVB, 75% to 95% PVB, 75% to 100% PVB, 80% to 90% PVB, 80% to 95% PVB, 80% to 100% PVB, 90% to 95% PVB, 90% to 100% PVB, or 95% to 100% PVB. In some cases, the dissolvable film can comprise about 1% PVB, 5% PVB, 10% PVB, 25% PVB, 50% PVB, 75% PVB, 80% PVB, 90% PVB, 95% PVB, or 100% PVB.

In some cases, a dissolvable film can comprise an ethanol soluble polymer in addition to or instead of PVB

A dissolvable film can comprise a polyvinyl alcohol (PVA) polymer. The dissolvable film can comprise 1-100% PVA. The dissolvable film can comprise at least 1% PVA. In some cases, the dissolvable film can comprise up to 100% PVA. In some cases, the dissolvable film can comprise 1% to 5% PVA, 1% to 10% PVA, 1% to 25% PVA, 1% to 50% PVA, 1% to 75% PVA, 1% to 80% PVA, 1% to 90% PVA, 1% to 95% PVA, 1% to 100% PVA, 5% to 10% PVA, 5% to 25% PVA, 5% to 50% PVA, 5% to 75% PVA, 5% to 80% PVA, 5% to 90% PVA, 5% to 95% PVA, 5% to 100% PVA, 10% to 25% PVA, 10% to 50% PVA, 10% to 75% PVA, 10% to 80% PVA, 10% to 90% PVA, 10% to 95% PVA, 10% to 100% PVA, 25% to 50% PVA, 25% to 75% PVA, 25% to 80% PVA, 25% to 90% PVA, 25% to 95% PVA, 25% to 100% PVA, 50% to 75% PVA, 50% to 80% PVA, 50% to 90% PVA, 50% to 95% PVA, 50% to 100% PVA, 75% to 80% PVA, 75% to 90% PVA, 75% to 95% PVA, 75% to 100% PVA, 80% to 90% PVA, 80% to 95% PVA, 80% to 100% PVA, 90% to 95% PVA, 90% to 100% PVA, or 95% to 100% PVA. In some cases, the dissolvable film can comprise about 1% PVA, 5% PVA, 10% PVA, 25% PVA, 50% PVA, 75% PVA, 80% PVA, 90% PVA, 95% PVA, or 100% PVA.

In some cases, the dissolvable film comprises a combination of a PVA, a polymer compatibilizer and a plasticizer (e.g., a polymer plasticizer). A polymer compatibilizer may be added to a dissolvable film to increase transparency in the film. A plasticizer may be added to a dissolvable film to promote plasticity and/or flexibility in the film.

Non-limiting examples of a polymer compatibilizer include cellulose ethers such as methylcellulose, carboxymethyl cellulose, hydroxypropyl methylcellulose, sodium carboxymethyl cellulose and modified starches such as acid-modified hydroxyethyl starches and hydroxypropylated starches.

Non-limiting examples of a sugar alcohol plasticizer include mannitol, isomalt, sorbitol, adonitol, pentaerythritol, xylitol, erythritol, dulcitol and maltitol. In some cases, a sugar alcohol plasticizer can be a combination of materials as described herein.

In such embodiments, the dissolvable film can comprise 10-30% by weight of a combination of a polymer compatibilizer and a sugar alcohol plasticizer. The dissolvable film can comprise about 2% to about 5%, about 2% to about 10%, about 2% to about 20%, about 2% to about 30%, about 2% to about 40%, about 5% to about 10%, about 5% to about 20%, about 5% to about 30%, about 5% to about 40%, about 10% to about 20%, about 10% to about 30%, about 10% to about 40%, about 20% to about 30%, about 20% to about 40%, or about 30% to about 40% of a polymer compatibilizer and a sugar alcohol plasticizer. The dissolvable film can comprise about 2%, about 5%, about 10%, about 20%, about 30%, or about 40% of a polymer compatibilizer and a sugar alcohol plasticizer.

In some cases, the dissolvable film can comprise at least 1%, 2%, 5%, 10%, 15%, 20% or 25% of polymer compatibilizer. In some cases, the dissolvable film can comprise at least 1%, 2%, 5%, 10%, 15%, 20% or 25% of a sugar alcohol plasticizer.

In some instances, the dissolvable film can comprise cellulose. The dissolvable film can comprise 1-100% cellulose. The dissolvable film can comprise at least 1% cellulose. In some cases, the dissolvable film can comprise up to 100% cellulose. In some cases, the dissolvable film can comprise 1% to 5% cellulose, 1% to 10% cellulose, 1% to 25% cellulose, 1% to 50% cellulose, 1% to 75% cellulose, 1% to 80% cellulose, 1% to 90% cellulose, 1% to 95% cellulose, 1% to 100% cellulose, 5% to 10% cellulose, 5% to 25% cellulose, 5% to 50% cellulose, 5% to 75% cellulose, 5% to 80% cellulose, 5% to 90% cellulose, 5% to 95% cellulose, 5% to 100% cellulose, 10% to 25% cellulose, 10% to 50% cellulose, 10% to 75% cellulose, 10% to 80% cellulose, 10% to 90% cellulose, 10% to 95% cellulose, 10% to 100% cellulose, 25% to 50% cellulose, 25% to 75% cellulose, 25% to 80% cellulose, 25% to 90% cellulose, 25% to 95% cellulose, 25% to 100% cellulose, 50% to 75% cellulose, 50% to 80% cellulose, 50% to 90% cellulose, 50% to 95% cellulose, 50% to 100% cellulose, 75% to 80% cellulose, 75% to 90% cellulose, 75% to 95% cellulose, 75% to 100% cellulose, 80% to 90% cellulose, 80% to 95% cellulose, 80% to 100% cellulose, 90% to 95% cellulose, 90% to 100% cellulose, or 95% to 100% cellulose. In some cases, the dissolvable film can comprise about 1% cellulose, 5% cellulose, 10% cellulose, 25% cellulose, 50% cellulose, 75% cellulose, 80% cellulose, 90% cellulose, 95% cellulose, or 100% cellulose. In some instances, the dissolvable film may comprise PVA and cellulose.

In some instances, the dissolvable film can comprise naturally occurring water-soluble polymers. Non-limiting examples of naturally occurring water-soluble polymers include xanthan gum, guar gum, water-soluble polymer derivatives such as hydroxypropylated starch and ethoxylated starch amongst others.

In some instances, the dissolvable film can comprise ethanol-soluble polymers. Non-limiting examples of ethanol-soluble polymers can include poly-isobutyl methacrylate, polyethylene glycol, and ethyl cellulose amongst others.

In some instances, the dissolvable film is edible, food-grade, cosmetic grade or pharmaceutical grade. The dissolvable film can be safe for direct contact with skin products.

In some instances, the dissolvable film can be used as a wipe, for example, as a cleansing wipe.

In some instances, the dissolvable film can be at least 3 cm×3 cm, 5 cm×5 cm, 10 cm×10 cm, 15 cm×15 cm, 20 cm×20 cm, 25 cm×25 cm, or 30 cm×30 cm. In some cases, the dissolvable film can be up to 10 cm×10 cm, 15 cm×15 cm, 20 cm×20 cm, 25 cm×25 cm, 30 cm×30 cm, 35 cm×35 cm, or 40 cm×40 cm. In some instances the dissolvable film can be a square, a rectangle, a circle, or another shape. Such a dissolvable film can have an area of at least 10 cm², 20 cm², 30 cm², 40 cm², 50 cm², 60 cm², 70 cm², 80 cm², 90 cm², 100 cm², 150 cm², 200 cm², 250 cm², or 300 cm². In some cases, such a dissolvable film can have an area of up to 20 cm², 30 cm², 40 cm², 50 cm², 60 cm², 70 cm², 80 cm², 90 cm², 100 cm², 150 cm², 200 cm², 250 cm², 300 cm², or 400 cm².

In some instances, the dissolvable film may be substantially transparent or translucent. For example, the film may be cast to a thickness of about 50 μm to 3 mm, 50 μm to 2.28 mm, 50 μm to 2 mm, 50 μm to 1 mm, 50 μm to 500 μm, 50 μm to 100 μm, 100 μm to 3 mm, 100 μm to 2.28 mm, 100 μm to 2 mm, 100 μm to 1 mm, 100 μm to 500 μm, 500 μm to 3 mm, 500 μm to 2.28 mm, 500 μm to 2 mm, 500 μm to 1 mm, 1 mm to 3 mm, 1 mm to 2.28 mm, 1 mm to 2 mm, 2 mm to 3 mm, 2 mm to 2.28 mm, or 2.28 mm to 3 mm.

In some cases, the dissolvable film can be a flat or textured sheet. The dissolvable film can be double-layered. The two layers can be attached on 1, 2, 3, 4, 5, 6, or more sides. In some cases, the number of sides which are attached can depend on the shape of the film. For example, a double-layered film which is triangular in shape can be attached on 1, 2, or 3 sides. A double-layered film which is rectangular in shape can be attached on 1, 2, 3, or 4 sides. A double-layered film which is a pentagon can be attached on 1, 2, 3, 4, or 5 sides. A film which is hexagonal in shape can be attached on 1, 2, 3, 4, 5, or 6 sides. In some cases, a double-layered film can have more than 6 sides, and can be attached on any or all of the sides. In some cases, a double-layered dissolvable film can form a mitten, glove, or pouch shape. The mitten, glove, or pouch can accommodate 1 finger, 2 fingers, 3 fingers, 4 fingers, at least a portion of a hand, or a complete hand. In such a case, a first layer can fit on top of the finger, fingers, or hand, while a second layer can fit on the bottom of the finger, fingers, or hand. The mitten, glove, or pouch may include a cavity and an opening providing access to the cavity, wherein the opening and/or the cavity are configured to receive at least a portion of a hand. In some cases, a double layered film can have 3, 4, 5, or more layers.

In some cases, the film can have a measured opacity of about 90%, 80%, 70%, 60%, 50%, 45%, 40%, 35%, 30%, 25%, 20%, or less, as determined by a colorimeter. In some cases, the film can be brown, white, or colorless.

In some instances, the dissolvable film is substantially soluble in a stabilizing or buffer solution at room temperature. The degree of hydrolysis of a dissolvable film in a stabilizing solution can be at least 50%. The degree of hydrolysis of a dissolvable film in a stabilizing solution can be at least 50%, 60%, 70%, 75%, 80%, 85%, 90%, 95%, 98% or 99%. The degree of hydrolysis of a dissolvable film can be about 100%. When partially hydrolyzed, the stabilizing solution can comprise vinyl acetate units.

In some instances, the degree of hydrolysis may be dependent on the temperature. For instance, the film can completely dissolve into the stabilizing solution at a temperature of 37° C. In some instances, a film when cast to a thickness of about 1.0-2.0 mm may be able to dissolve in a stabilizing solution in less than 2 minutes at a temperature between 20° C.-40° C. In some cases, a 1.0-2.0 mm thick film may be able to dissolve in a stabilizing solution in less than 40 seconds, less than 50 seconds, less than 1 minute, less than 2 minutes or less than 5 minutes at a temperature between 20° C.-40° C.

### Solutions to Preserve and Stabilize a Biological Sample

The dissolvable films discussed herein are preferably dissolved or substantially dissolved in order to stabilize/preserve a biological sample. This can occur by taking the film (e.g., a wipe) and placing it in a stabilizing or buffer solution.

The dissolvable film comprising a biological sample may be dissolved or substantially dissolved in a stabilizing solution forming a solubilized sample composition. Such dissolution can result in a solubilized sample composition comprising the stabilizing solution, the dissolved film and the biological sample.

The stabilizing solution can comprise one or more components or reagents. In one instance, a stabilizing solution can comprise a stabilizing reagent. A stabilizing reagent may comprise a preservative reagent, chelating agents, chaotropic agents, organic solvents, surfactants, protein additives, ion exchange agents, reducing agents, oxidizing agents, free radical scavengers or a combination thereof. In some cases, a stabilizing agent can be a preservative agent, which can comprise chelating agents, chaotropic agents, organic solvents, surfactants, protein additives, ion exchange agents, reducing agents, oxidizing agents, free radical scavengers or a combination thereof. The stabilizing solution can comprise at least about 0.01%, 0.05%, 0.1%, 0.5%, 1%, 2%, 3%, 5%, 7%, 8.5%, 10%, 12.5%, 15%, 17% or 20% stabilizing reagents by volume. Examples of preservative reagents include, but are not limited to, sodium azide, streptomycin sulfate, parabens, ethylene glycol monophenyl ether and polyethylene glycol.

A preservative or a stabilizing reagent can be a high salt reagent. Non-limiting examples of salts include lithium chloride, sodium chloride, ethylenediaminetetraacetic acid (EDTA) disodium salt dihydrate, sodium citrate trisodium salt dihydrate, ammonium sulphate, sodium chloride, sodium thioglycolate, sodium hydrogen phosphate, guanidine isothiocyanate or sodium citrate. The stabilizing solution can have a combination of salts. The stabilizing solution can comprise at least about 0.01%, 0.05%, 0.1%, 0.5%, 1%, 2%, 3%, 5%, 7%, 8.5%, 10%, 12.5%, 15%, 17% or 20% salt by volume. The stabilizing solution can comprise a combination of salts. The stabilizing solution can comprise at least about 1%, 5%, 10%, 15%, 20% or 30% of a combination of salts by volume.

In some cases, a high salt preservative or stabilizing reagent can comprise a high amount of magnesium chloride. A high salt preservative or stabilizing reagent can comprise one type of salt (e.g., sodium chloride or magnesium chloride) or more than one type of salt (e.g. sodium chloride and magnesium chloride). A high salt preservative or stabilizing reagent can be salt saturated or substantially salt saturated such that additional salt may not significantly dissolve.

A high salt preservative or stabilizing reagent can comprise disodium EDTA. In some cases, a high salt preservative or stabilizing reagent can comprise at least 0.05 M, 0.1 M, 0.15 M, 0.2 M, 0.25 M, 0.3 M, or 0.35 M disodium EDTA. In some cases, a high salt preservative or stabilizing reagent can comprise no more than 0.2 M, 0.25 M, 0.3 M, 0.35 M, 0.4 M, 0.45 M, or 0.5 M disodium EDTA.

A high salt preservative or stabilizing reagent can comprise at least 5%, 10%, 15%, 20%, 25%, 30%, 35%, 40%, 45%, or 50% DMSO. In some cases, a high salt preservative or stabilizing reagent can comprise no more than 5%, 10%, 15%, 20%, 25%, 30%, 35%, 40%, 45%, or 50% DMSO.

In some cases, a high salt stabilizing reagent can be a DESS (DMSO-disodium EDTA-saturated salt) reagent. For example, a DESS reagent can comprise 0.25 M disodium EDTA, 20% DMSO, and saturated sodium chloride.

In some cases, a high salt stabilizing reagent can dissolve a film comprising PVA or cellulose. In some cases, a film comprising PVA or cellulose can dissolve in at least about 1 mL, 5 mL, 10 mL, 15 mL, 20 mL, 25 mL, 30 mL, 35 mL, 40 mL, 45 mL, or 50 mL of high salt stabilizing reagent. In some cases, a water-based reagent can dissolve a film comprising PVA or cellulose. In some cases, a film comprising PVA or cellulose can dissolve in at least about 1 mL, 5 mL, 10 mL, 15 mL, 20 mL, 25 mL, 30 mL, 35 mL, 40 mL, 45 mL, or 50 mL of a water-based reagent.

The stabilizing solution may be an organic solvent based solution. Examples of organic solvent based solutions contemplated herein include, but are not limited to, buffers including ethanol, methanol, DMSO, etc. In some instances, a stabilizing solution may be a water-based solution.

A buffer or stabilizing solution can be or comprise an ethanol-based preservative or stabilizing reagent. An ethanol-based preservative or stabilizing reagent can comprise a high amount of ethanol. In some cases, an ethanol-based preservative or stabilizing reagent can comprise at least 65%, 70%, 75%, 80%, 85%, 90%, 95%, 99%, or 100% ethanol. In some cases, an ethanol-based preservative or stabilizing reagent can comprise no more than 70%, 75%, 80%, 85%, 90%, 95%, or 99% ethanol. An ethanol-based preservative or stabilizing reagent can comprise between 70% and 100%, between 80% and 100%, between 90% and 100%, between 70% and 99%, between 75% and 99%, between 80% and 99%, between 85% and 99%, between 90% and 99%, between 95% and 99%, between 75% and 95%, between 80% and 95%, between 85% and 95%, or between 90% and 95% ethanol. In some cases, an ethanol-based preservative or stabilizing reagent can additionally comprise disodium ethylenediaminetetraacetic acid (EDTA), dimethyl sulfoxide (DMSO), magnesium chloride, and/or sodium chloride.

In some cases, an ethanol-based stabilizing reagent can dissolve a film comprising PVB. In some cases, a film comprising PVB can dissolve in at least about 1 mL, 5 mL, 10 mL, 15 mL, 20 mL, 25 mL, 30 mL, 35 mL, 40 mL, 45 mL, or 50 mL of an ethanol-based stabilizing reagent.

A buffer or stabilizing solution comprising a preservative or stabilizing reagent can be at least 5%, 10%, 20%, 30%, 40%, 50%, 60%, 70%, 80%, 90%, or 100% preservative or stabilizing reagent by volume.

In some cases, the preservative or stabilizing reagent can be ethanol. In some cases, an ethanol based preservative or stabilizing reagent can be at least about 70%, 80%, 90%, 95%, or 100% ethanol. In some cases, an ethanol based preservative or stabilizing reagent can be no more than about 70%, 80%, 90%, 95%, or 100% ethanol.

In some cases, a buffer solution comprising a stabilizing solution can be water based. In such cases, a film comprising PVA or cellulose can be dissolved in such a stabilizing solution.

In some cases, a stabilizing solution comprising an ethanol stabilizing solution can be ethanol based. In such cases, a film comprising PVB can be dissolved in such a stabilizing solution.

In some instances, a stabilizing or buffer solution is one of OMNIgene®.GUT, RNAlater™, Longmire buffer, or Cary-Blair medium.

In some instances, the stabilizing may comprise additional components such as tris hydroxymethyl aminomethane, TCEP-HCl, N-octylpyridinium bromide solution, antiseptic reagents or a combination thereof. The stabilizing solution can comprise up to 1%, 5%, 10%, 15%, 20% or 30% of additional components by volume.

A stabilizing or buffer solution can have a range of pHs. In some instances, a stabilizing solution has a pH of at least or at most 4, 4.5, 5.0, 5.5, 5.9, 6.0, 6.2, 6.5, 6.8, 7.0, 7.2, 7.4, 7.6, 7.8, 8.0, 8.2, 8.4, 8.6, 8.8, 9.0, 9.2, 9.5, 9.7, 10.0, 10.2, 10.5, 10.7, 11.0, 11.2, 11.5, 11.7 or 12.0. The pH of the stabilizing may be adjusted using acidic or basic reagents.

The stabilizing or buffer solution may be able to effectively stabilize a biological sample (e.g., macromolecules such as nucleic acids, metabolites, proteins, cells and cellular components). Effective stabilization can provide for maintenance of sample integrity such that, e.g., at least about 50% of the initial amount of the biological sample contained is still present after storing the sample at room temperature or freezing temperatures for a specified or predetermine period of time. For instance, macromolecules can be stable at room temperature in the stabilizing solution for at least 1 day, 5 days, 10 days, 15 days, 20 days or at least 30 days. Macromolecules can be stable at −20° C. in the stabilizing solution for at least 10 days, 20 days, 1 month, 2 months, 3 months or 5 months. Macromolecules may be stable at −80° C. in the stabilizing solution for at least 15 days, 30 days, 2 months, 3 months, 5 months, 10 months or up to a year.

Maintaining the sample integrity in the solubilized sample composition may or may not require freezing the composition. The solubilized sample composition comprising the stabilizing solution and the biological sample may not need to be frozen before any further processing. In some cases, the solubilized sample composition may be frozen once before further processing. In some cases, the solubilized sample composition may be stable upon freezing. In some cases, the solubilized sample composition may be frozen and thawed multiple times before processing. Multiple freeze thaw cycles may be performed to lyse cells in the solution.

The dissolvable film including the biological sample can be dissolved in to a stabilizing solution by a subject. The dissolvable film can be dissolved in to the stabilizing solution using mechanical dissociation methods such as vortexing, shaking, rocking, invert-mixing, rotating, and/or soaking. In some cases, no additional mechanical dissociation methods are needed to dissolve the film in to the stabilizing solution and soaking of the film comprising the biological sample may be sufficient to dissolve or substantially dissolve the film.

In some instances, the sealable container can be any conventionally used sealable container that does not leak or substantially leak during transportation after sealing. Non-limiting examples of sealable containers include vials, tubes, centrifuge tubes, collection tubes, Falcon tubes, Sarstedt fecal collection tubes and other suitable containers.

A capacity of the sealable container can be at least 1 ml. A capacity of the sealable container may be greater than 1 ml, 5 ml, 10 ml, 20 ml, 50 ml, 100 ml or 150 ml. A capacity of the sealable container may be at least 1 ml, 5 ml, 10 ml, 20 ml, 50 ml, 100 ml, 150 ml or 200 ml. A capacity of the sealable container may be at most 1 ml, 5 ml, 10 ml, 20 ml, 50 ml, 100 ml, 150 ml or 200 ml.

In some cases, a sealable container can comprise a volume of buffer or stabilizing solution of at least 1 ml. A volume of a stabilizing solution can be at least about 1 ml, 5 ml, 10 ml, 20 ml, 50 ml, 100 ml or 150 ml. A volume of a stabilizing solution can be no more than about 1 ml, 5 ml, 10 ml, 20 ml, 50 ml, 100 ml, 150 ml or 200 ml. A volume of a stabilizing reagent can be at least about 1 ml, 5 ml, 10 ml, 20 ml, 50 ml, 100 ml or 150 ml. A volume of a stabilizing reagent can be no more than about 1 ml, 5 ml, 10 ml, 20 ml, 50 ml, 100 ml, 150 ml or 200 ml.

A volume of buffer or stabilizing solution can dissolve a film. In some cases, a volume of a buffer solution or a stabilizing reagent can dissolve a film which is at least 3 mm×3 mm, at least 5 mm×5 mm, at least 10 mm×10 mm, or at least 40 mm×40 mm.

A volume of buffer or stabilizing solution can dissolve a film over a period of at most about 10 seconds, 30 seconds, or 60 seconds. A volume of buffer or stabilizing solution can dissolve a film over a period of at most about 1 minute, 5 minutes, 10 minutes, 30 minutes, or 60 minutes.

In some instances, the solubilized sample composition comprising the stabilizing solution, the biological sample and the dissolved dissolvable film may comprise intact cells from the biological sample. The solubilized sample composition may also comprise non-intact cells. In some cases, intact cells are not significantly perturbed by the dissolving of the wipe.

### Sample Processing

The methods and systems presented herein may be used for identifying a microbiome or metabolite population from a solution. The identification of a microbiome population may comprise performing a nucleic acid content analysis on a biological sample. The biological sample can be in a solubilized sample composition. The solubilized sample composition can comprise a stabilizing solution including the biological sample and a dissolved dissolvable film. The method may comprise identifying at least one microbe present in the solubilized sample composition based on the nucleic acid content analysis. The method may comprise determining a microbiome profile of the biological sample.

Determining the microbiome profile of the biological sample can comprise identifying at least one microbe. Determining the microbiome profile of the biological sample can comprise the identification of at least five microbes, at least 10 microbes, at least 20 microbes, at least 30 microbes, at least 50 microbes, at least 100 microbes, at least 200 microbes or at least 500 microbes. Determining the microbiome profile of the biological sample can comprise identifying a microbial genus or a species present in the biological sample. Determining the microbiome profile of the biological sample can comprise the identification of at least one genus, at least five genera, at least 10 genera, at least 20 genera, at least 30 genera, at least 50 genera, at least 100 genera, at least 200 genera or at least 500 genera.

Determining the microbiome profile of a biological sample may comprise analysis of the nucleic acid content. In some instances, the nucleic acid content analysis comprises sequencing the nucleic acid content of the biological sample. Sequencing the nucleic acid content can comprise next-generation sequencing, long read sequencing, Sanger sequencing, high-throughput sequencing, fluorescence based sequencing, polymerase chain reaction based sequencing or nanopore sequencing.

In some instances, sequencing the nucleic acid content comprises sequencing the whole genome of a microbe in the biological sample. Sequencing the nucleic acid content can comprise sequencing the 16S sequences for the identification of bacteria. In some cases, sequencing the V1, V2, V3, V4 or V5 regions or combinations thereof of 16S sequences in the nucleic acid content may be performed. Sequencing the nucleic acid content can comprise sequencing the ITS1 sequence region for the identification of fungi. Sequencing the nucleic acid content can comprise sequencing the 18S sequences for the identification of eukaryotic microbes. In some cases, sequencing the V4 region of 18S sequences in the nucleic acid content may be performed.

In some cases, analysis of the nucleic acid content may require purification. The purification of nucleic acid content can be performed using a nucleic acid purification kit. The nucleic acid purification kit may be any conventional nucleic acid purification kit, for instance a DNA purification kit or a RNA purification kit.

The methods and systems presented herein may be used for identifying a metabolite population from a solution. The identification of a metabolite population may comprise performing a protein or lipid content analysis on a biological sample. The method may comprise identifying at least one metabolite present in the solubilized sample composition based on the protein or lipid content analysis. The method may comprise determining a metabolite profile of the biological sample.

In some instances, determining the metabolite profile of the biological sample can comprise identifying at least one metabolite. Determining the metabolite profile of the biological sample can comprise the identification of at least five metabolites, at least 10 metabolites, at least 20 metabolites, at least 30 metabolites, at least 50 metabolites, at least 100 metabolites, at least 200 metabolites or at least 500 metabolites.

Determination of the microbiome or metabolite population in a biological sample may be performed by analyzing the protein or lipid content of the biological sample. In some cases, the protein or lipid content can be purified. The purification of protein or lipid content can be performed using a protein or lipid purification kit. The protein or lipid purification kit may be any conventional protein or lipid purification kit.

In some cases, identifying the microbiome or metabolite population may not comprise freezing and thawing the biological sample or the solubilized sample composition including the biological sample. Alternatively, the identification of the microbiome or metabolite population may comprise one or more freezing and thawing steps.

### Sampling Kits

One or more systems for analyzing a biological sample are presented herein. The one or more systems may comprise a sampling kit. In some aspects, a sampling kit that may be used to collect and preserve a biological sample is presented herein. A sampling kit may comprise a dissolvable film for the collection of a biological sample. The dissolvable film can be a water-soluble film or an ethanol-soluble film as described previously herein. The kit may also comprise a sealable container for sample collection. The kit can comprise a buffer or stabilizing solution. In some cases, the sealable container can comprise a buffer or stabilizing solution. A dissolvable film in a kit can be soluble in a stabilizing solution in the same kit. In some cases, a kit can comprise a preservative or stabilizing reagent. The preservative or stabilizing reagent can be pre-mixed with the buffer or stabilizing solution, or can be separate from the buffer or stabilizing solution. A preservative or stabilizing reagent in a kit can be soluble or miscible in a buffer or stabilizing solution in the same kit.

In some cases, the water-soluble film in a kit can comprise PVA or cellulose as described herein. In such cases, the kit can comprise a water-based buffer or stabilizing solution. In some cases, the kit can comprise DESS.

In some cases, the ethanol-soluble film in a kit can comprise PVB as described herein. In such cases, the kit can comprise an ethanol-based stabilizing solution. In some cases, the kit can comprise 95% ethanol.

The buffer or stabilizing solution may be packaged in the sealable container. A subject can use a dissolvable film to collect a biological sample and then place the film into the sealable container comprising the stabilizing solution. Alternatively, the stabilizing solution may be packaged separately. A subject can use the dissolvable film to collect a sample, such as a biological sample. The separately packaged stabilizing solution can be emptied in to the sealable container in the kit. The dissolvable wipe with the sample can then be dissolved in to the stabilizing solution in the sealable container.

The solubilized sample composition can be transported or delivered to a laboratory or a testing center. In some instances, the sealable container is transported or delivered at room temperature. Alternatively, the sealable container is transported or delivered on dry ice, in an ice box, in a cool box, or with an ice pack.

The kit and the sealable container may be uniquely identifiable. In some cases, the kit and the sealable container are barcoded. The barcode may be used to link the biological sample to a subject.

In some instances, the kit comprises materials for one sample collection. Alternatively, the kit can comprise materials for multiple sample collections. The dissolvable film can be packaged dry. Alternatively, the dissolvable film can be packaged in a solution that does not dissolve the film and keeps it stable in the package.

In some instances, the sampling kit can be used by a subject for personal use. For instance, a person may collect the sample themselves and send it to a laboratory for diagnosis. Alternatively, the sampling kit may be used in a laboratory or clinic or hospital setting where the kit materials are provided to a subject or to collect a sample from a subject by a trained professional. In some cases, the dissolvable film can be individually packaged. The dissolvable film can be in a sealed packet or a sachet. Alternatively, the dissolvable film can be provided as a stacked or rolled set of sheets in a sealed package.

The one or more systems may comprise one or more sample processing modules. A sample processing module can be configured to identify at least one microbe or metabolite in the biological sample.

The one or more sample processing modules may be configured to perform assays for the identification of a microbiome or metabolite population. Non-limiting examples of such assays or processes include polymerase chain reaction (PCR), real time PCR (RT-PCR), quantitative real time PCR (qRT-PCR), sequencing, ligase chain reaction, nucleic acid library generation, strand displacement amplification, genotyping, ELISA, microarrays, fluorometric assays, mass spectroscopy, HPLC or other assays. The one or more sample processing modules can comprises an immunoassay, a flow cytometer, a microchip, a sequencer, LC/MS, GC/MS or NMR.

The methods and systems of identification of microbiome can comprise identification of microorganisms such as bacteria, archaea, fungi and viruses. The methods and systems of identification of microbiome can comprise identification on a genus level. Non-limiting examples of genera of microbes that can be identified include Bacteroides, Sutterella, Faecalibacterium, Parabacteroides, Phascolarctobacterium, Dialister, Flavonifractor, Lachnospira, Blautia, Megasphaera, Coprococcus, Anaerostipes, Coprobacillus, Catenibacterium, Dorea, Hungatella, Holdemanella, Erysipelatoclostridium, Eisenbergiella, Escherichia, Lachnoclostridium, Fusobacterium, Bifidobacterium, Anaerotruncus, Streptococcus, Prevotella, Subdoligranulum, Alloprevotella, Megamonas, Roseburia, Barnesiella, Desulfovibrio, Alistipes, Ruminococcus and Odoribacter amongst others. In some cases, microbes can be identified on a phylum level. Non-limiting examples of phyla that can be identified include Bacteroidetes, Firmicutes, Proteobacteria, Verrucomicrobia, Actinobacteria, Fusobacteria, and Cyanobacteria amongst others. In some cases, microbes can be identified on a species level.

The methods and systems of identification of microbiome can comprise identification of metabolites in a biological sample retrieved from a subject. The metabolites may be from various sources, for instance, gut, skin, oral and fecal metabolites. Non-limiting examples of metabolites include bile acids, such as cholic acid, deoxycholic acid, and lithocholic acid, methylamines such as trimethylamine (TMA), trimethylamine-N-oxide (TMAO), polyphenolics such as catechin, enterodiol and enterolactone, indoles such as 3-methylindole, short chain fatty acids such as acetate, butyrate and propionate, polyamines, such as spermidine, spermine and putrescine and vitamins such as vitamin B12, folate, etc.

In some cases, determination of the microbiome or metabolite profile may lead to diagnosis of a disease or a condition in the subject. The subject can host a variety of microorganisms and metabolites. The subject can have different microbiomes and metabolites in various habitats on and in their body. The subject may be diagnosed or suspected of being at high risk for a disease. The subject may have a microbiome or metabolite state that is contributing to a disease. In some cases, the subject is not necessarily diagnosed or suspected of being at high risk for the disease. In some instances a subject may be suffering from an infection or at risk of developing or transmitting to others an infection.

Determination of the microbiome and metabolite population of a biological sample may be followed by generating a report. A report may be generated after the identification of at least one microbe or one genus present in the microbiome population of the biological sample. In some cases, a report may be generated after the identification of at least one type of metabolite present in the biological sample. The report may also comprise information about the subject. In some cases, the report may comprise a diagnosis of a particular condition or disease.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLE

### Example 1

### Species Composition and Abundance of Microbiome in Stool Sample

Four human stool samples were collected. Fecal samples 1 and 3 were collected using water-soluble film as described herein. Fecal samples 2 and 4 were control samples collected using a conventional fecal sample collection method, using a toilet hat and spoon. No film was used for samples 2 and 4. Examples of such a method include those used by Genotek, Zymo, and BGI. The water-soluble film was used as the subject would use toilet paper. Each water-soluble film was dissolved in a stabilizing solution. Fecal samples 1 and 2 were dissolved in a stabilizing solution comprising DESS (DMOS/EDTA/Saturated Salts) DNA preservative reagent. Fecal samples 3 and 4 were dissolved in a stabilizing solution comprising BGI's DNA preservative reagent (N-octylpyridinium bromide-based reagent; see Han et al. Microbiome (2018) 6:43).

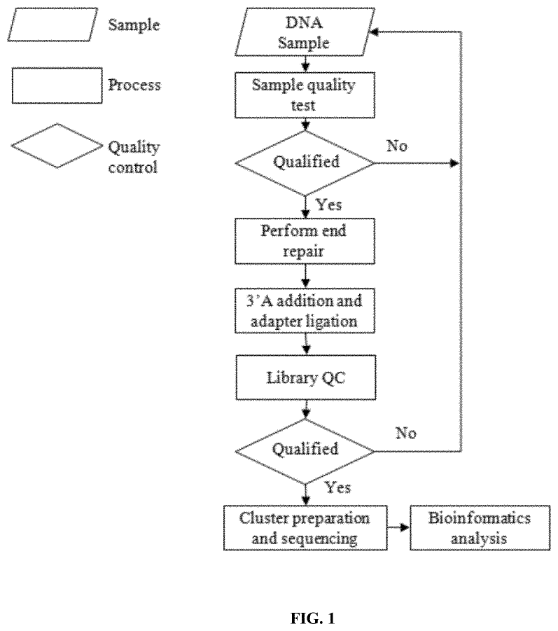

After mixing the fecal sample with a stabilizing solution comprising a preservative reagent, each sample was sent to a laboratory. DNA was extracted from the fecal samples using a standard method. Sample quality was tested. If the sample quality was not sufficient, DNA was extracted again. If the sample quality was sufficient, end repair was performed, and 3′ A addition and adapter ligation was performed. The quality of the resulting DNA library was checked. If the quality was not good, the DNA was again extracted, and the process repeated. If the quality was good, cluster preparation and sequencing was performed, and bioinformatics analysis was performed. A flow chart describing this process can be found in FIG. 1.

Taxonomic composition of each fecal sample was determined and displayed as a histogram. Histograms of each sample are shown at the phylum, class, and order level, separately. The ratio of each species in a certain sample is directly displayed.

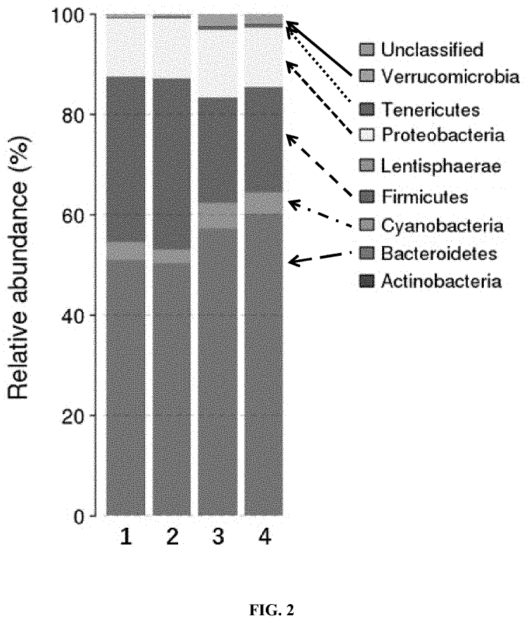

At the phylum level, all species were used to produce the histogram. The taxonomic composition distribution in samples at the phylum level is shown as percent relative abundance in FIG. 2. Actinobacteria, Bacteroidetes, Cyanobacteria, Firmicutes, Lentisphaerae, Proteobacteria, Tenericutes, Verrucomicrobia, and unclassified microbes were present in the samples. The results of the sample collected using the wipe were similar to the results of the samples collected using the conventional fecal sample collection method.

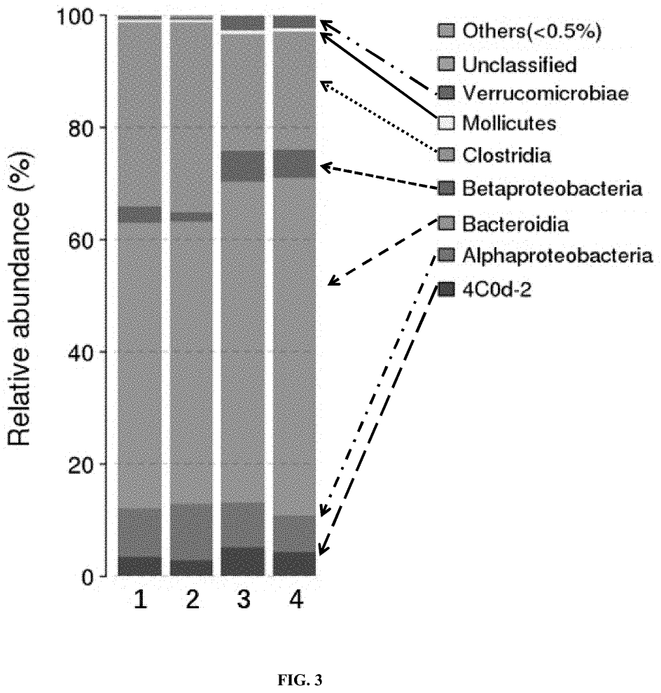

FIG. 3 displays the taxonomic composition distribution in samples at the class level as percent relative abundance. Species having abundance less than 0.5% in all samples were classified as “others.” 4C0d-2, alphaproteobacteria, bacteroidia, betaproteobacteria, clostridia, mollicutes, verrucomicrobiae, unclassified, and other microbes were present in the samples. The results of the sample collected using the wipe were similar to the results of the samples collected using the conventional fecal sample collection method.

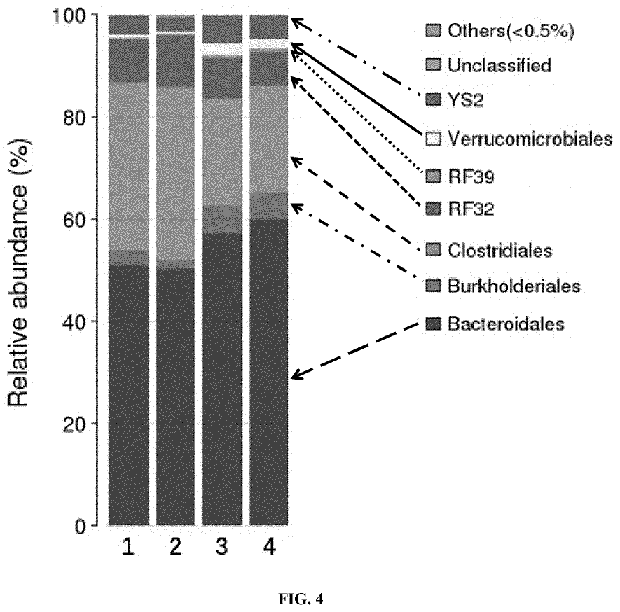

FIG. 4 displays the taxonomic composition distribution in samples at the order level as percent relative abundance. Species having abundance less than 0.5% in all samples were classified as “others.” Bacteroidales, burkholderiales, clostridiales, RF32, RF39, verrucomicrobiales, YS2, unclassified, and other microbes were present in the samples. The results of the sample collected using the wipe were similar to the results of the samples collected using the conventional fecal sample collection method.

1. A method of preserving a biological sample, the method comprising:
a. collecting a biological sample on a dissolvable film; and
b. dissolving the dissolvable film including the biological sample in a stabilizing solution, wherein the stabilizing solution is disposed in a sealable container.

2. The method of claim 1, further comprising analyzing the biological sample.

3. The method of claim 1, wherein the biological sample comprises a microbiome population.

4. The method of claim 1, wherein the biological sample comprises a metabolite population.

5. The method of claim 1, wherein the biological sample comprises a fecal, skin, vaginal, ear, or oral sample.

6. The method of claim 1, wherein the dissolvable film is water soluble.

7. The method of claim 1, wherein the dissolvable film is ethanol soluble.

8. The method of claim 1, wherein the dissolvable film comprises a polyvinyl butyral polymer, a polyvinyl alcohol polymer, or cellulose.

9. (canceled)

10. (canceled)

11. The method of claim 1, wherein the dissolvable film comprises (i) a combination of a polyvinyl butyral polymer, a polymer compatibilizer, and a plasticizer or (ii) a combination of a polyvinyl alcohol polymer, a polymer compatibilizer, and a sugar alcohol plasticizer.

12. (canceled)

13. The method of claim 11, wherein the polymer compatibilizer comprises cellulose ether polymer, sodium carboxymethyl cellulose, modified starch, or a combination thereof.

14. The method of claim 11, wherein the dissolvable film comprises from 10% to 30% by weight of a polymer compatibilizer and a sugar alcohol plasticizer.

15.-20. (canceled)

21. The method of claim 1, wherein the dissolvable film is substantially soluble in the stabilizing solution below 37° C.

22. (canceled)

23. The method of claim 1, wherein the stabilizing solution comprises a preservative or a stabilizing reagent.

24.-27. (canceled)

28. The method of claim 1, wherein a volume of the sealable container is from 5 ml to 150 ml.

29. (canceled)

30. The method of claim 1, wherein the stabilizing solution stabilizes the biological sample at room temperature for up to 15 days.

31.-33. (canceled)

34. The method of claim 1, further comprising transporting or delivering the sealable container comprising the stabilizing solution and the biological sample to a laboratory or a testing center.

35. The method of claim 34, wherein the sealable container is transported or delivered at room temperature.

36. The method of claim 34, wherein the sealable container is transported or delivered on dry ice, in an ice box, in a cool box, or with an ice pack.

37. The method of claim 1, wherein the sealable container comprising the stabilizing solution and the biological sample is not frozen before any further processing.

38. The method of claim 1, wherein the surface area of the dissolvable film is from 3 cm×3 cm to 40 cm×40 cm.

39.-72. (canceled)
